(19) United States
(12) Patent Application Publication
(10) Pub. No.: US 2006/0154268 A1
(43) Pub. Date: Jul. 13, 2006

(54) Circadian rhythm control gene cluster, DNA chip, and methods for using the DNA chip

(76) Inventors: Takuro Yamamoto, Kanagawa (JP); Haruhiko Soma, Tokyo (JP); Takayoshi Mamine, Kanagawa (JP); Toru Takumi, Kyoto (JP); Yasukazu Nakahata, Osaka (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/174,992

(22) Filed: Jul. 5, 2005

(30) Foreign Application Priority Data
Jul. 6, 2004 (JP) 2004-199122

(51) Int. Cl. C12Q 1/68; C07H 21/04; C12M 1/34

(52) U.S. Cl. 435/6; 435/287.2; 536/23.5

(57) ABSTRACT

A circadian rhythm control gee cluster (Bmal1 gene, Npas2 gene, Rev-erbα, Dbp gene, Per3 gene, Per2 gene and Per1 gene) having a given expression timing and expression sequence under normal conditions is provided along with a DNA chip having, at least, the circadian control gene group sequentially arranged. There are also provided a method for predicting modulation in expression timing of the circadian rhythm control gene cluster, a method for detecting a circadian rhythm modulation, a method for selecting a circadian rhythm regulator, a method for screening a circadian rhythm regulator, and screening of a substance involving modulation of a circadian rhythm as a side effect, each using the DNA chip.

### BACKGROUND OF THE INVENTION

This invention relates to a circadian rhythm control gene cluster, a DNA chip wherein at least the circadian rhythm control gene cluster is sequenced, a method for predicting a modulation of expression timing of a circadian rhythm control gene cluster by use of the DNA chip, a method for detecting modulations of circadian rhythms, a method for selecting a circadian rhythm regulator, and a screening method.

Circadian rhythms are biorhythms with a periodic cycle of about 24 hours, and are an in vivo phenomenon that is universally observed for many organisms covering from unicellular organisms to human beings. Individual organisms have genes related to the control of circadian rhythms (which may be hereinafter referred to as “circadian rhythm control gene”), which serve to adjust the circadian rhythms.

With mammals, an expressed amount of the circadian rhythm control genes is much higher at nucleus suprachiasmatica (SCN) of the brain's hypothalamus region, which serves as a center of adjusting the circadian rhythms at an individual level. On the other hand, the circadian rhythm control genes are expressed in other in-vivo tissues (i.e. circadian rhythm peripheral tissues) where the circadian rhythms are adjusted every cell and every tissue by means of circadian rhythm control genes like the circadian rhythm center. Besides, it has been accepted that there seems to be a mechanism where the circadian rhythms at an individual level based on the circadian rhythm center integrate the circadian rhythms of the circadian rhythm peripheral tissues.

For the circadian rhythm control genes of mammals, there are known Per genes (Per1, Per2, Per3), Clock gene, Bmal genes (Bmal1, Bmal2, Bmal3), Cry gene, Npas2 gene, Dbp gene, Rev-erb genes (Rev-erbα, Rev-erbβ and the like (hereinafter referred generically to “circadian rhythm control gene cluster).

In a code region or upstream sequence of a circadian rhythm control gene, there exists a transcriptional control region (i.e. a specific base sequence) of the circadian rhythm control gene. For the transcriptional control region, RORE, a DBP binding site and E-BOX have been clarified. The transcriptional control region is a binding region of transcription factor, and the transcription factor is bound to a transcriptional control region and controls transcription and expression of circadian rhythm control genes.

RORE exits in a code region of Bmal1 or an upstream sequence thereof. It is known that the Ror transcription factor family (REV-ERB and the like) that is a transcription factor binds to RORE to adjust the expression of Bmal1 gene. It will be noted that REV-ERB is a transcription product (protein) of the Rev-erb gene.

The DBP binding region exists in a code region or an upstream sequence thereof such as of Per1 gene. DBP (i.e. a transcription product of Dbp gene) etc., which are a transcription factor, bind to a DBP binding site, thereby promoting the expression of Per1 gene.

EBOX exists in a code region or an upstream sequence thereof such as of Per1 gene and Per2 gene. CLK-BMAL complex (a complex of a transcription product of Clock gene and a transcription product of Bmal gene) that is a transcription factor binds to E-BOX to promote expression of Per1 gene and Per2 gene.

As having set out hereinabove, the expression of each circadian rhythm control gene is adjusted based on a transcription product (protein) of the circadian rhythm control gene. It will be noted that in “Molecular Biology of Clock Genes” edited by Hitoshi Okamura and Yoshitaka Fukada and published by Springer-Verlag Tokyo K.K. Apr. 5, 2004), such circadian rhythm control genes and transcriptional control regions (E-BOX, DBP binding site, RORE) as stated above are described.

### SUMMARY OF THE INVENTION

It is considered that circadian rhythm formation of cell unit is controlled by expression timings of the circadian rhythm control gene cluster. Thus, it is important to clarify the expression timing and sequence of individual circadian rhythm genes. More particularly, the clarification of the expression timing and sequence of the circadian rhythm control genes leads to assistance of elucidation of the circadian rhythm control mechanism.

Further, when the expression timing and seqence of the circadian rhythm control gene cluster under normal conditions are clarified, one is enabled to readily detect a modulation of circadian rhythm in view of a deviation of the expression timing and sequence of the circadian rhythm control gene cluster.

In addition, the clarification of the expression timing and sequence of a circadian rhythm control gene cluster under normal conditions makes it possible to search for substances capable of shifting back and forth the expression timing and sequence of the circadian rhythm control gene cluster.

Accordingly, it is an object of the invention to provide an expression timing and sequence of a circadian rhythm control gene cluster under normal conditions.

It is another object of the invention to provide a method for detecting a modulation of circadian rhythms by use of the expression timing and sequence.

It is a further object of the invention to provide a screening method using the expression timing and sequence.

We have found for the first time that in substantially all circadian rhythm peripheral tissues, the expression timings of the following circadian rhythm control gene cluster (1) to (7) indicate circadian changes. It has been also found that the expression sequences of these circadian rhythm control gene clusters under normal conditions appear regularly. In addition, we have found the location and number of transcription control regions (RORE, DBP binding site, and E-BOX) related to the circadian rhythm control genes for the first time.

In the practice of the invention, when standardized on 16 o'clock in terms of circadian time (e.g. a.m. 0 o'clock of an ordinary time when a light conditions-initiating time is taken as 8 o'clock of an ordinary time), there is provided a circadian rhythm control gene cluster in which after expression of the following genes (1) or/and (2), the following genes (3) to (7) start to express sequentially and which contains all the genes (1) to (7) or a plurality of genes selected from the genes (1) to (7):
(1) Bmal1 gene having RORE in a code region or an upstream sequence thereof;
2( ) Npas2 gene having RORE in an upstream sequence of a code region;
3( ) Rev-erbα gene having RORE, DBP binding site and E-BOS in a code region or an upstream sequence thereof;
4( ) Dbp gene having RORE and E-BOX in a code region or an upstream sequence thereof;
5( ) Per3 gene having a DBP binding site in a code region or an upstream sequence thereof;

6( ) Per1 gene having a DBP binding site in an upstream sequence of a code region; and
7( ) Per2 gene having E-BOX in an upstream sequence of a code region.

As stated hereinabove, when standardized on 6 o'clock in terms of circadian time, the circadian rhythm control gene cluster expresses in the following sequence of:
(a) a gene having RORE in a code region or an upstream sequence thereof;
(b) a gene having a DBP binding site in a code region or an upstream sequence thereof; and
(c) a gene having E-BOX in a code region or an upstream sequence thereof.

More particularly, among the three transcription control regions (RORE, DBP binding site and E-BOX), Bmal1 and Npas2 having RORE alone initially express, after which Rev-erbα having, aside from RORE, DBP binding site and R-BOX expresses, followed by expression of Dbp having E-BOX aside from RORE (a). Next, Per3 having the DBP binding site alone expresses, and then Per1 having E-BOX aside from DBP binding site (b). Thereafter, Per2 having E-BOX alone expresses (c).

It is to be noted that the base sequences of genes (1) to (7) have been laid open as the public database of NCBI (National Center for Biotechnology Information). The gene numbers of the respective genes in the database of NCBI are indicated below wherein each figure in parentheses indicates a region in gene. (1) Bmall ; human NT_{—}009237 (12054318 to 12069318), mouse NT_{—}081129.1 (107781 to 122781), rat NW_{—}047562.1 (13774073 to 13789073). (2) Npas2: human hCG27614 (95632226 to 65646226), mouse mCG8437 (35980102 to 35994102), rat rCT22431 (39204499 to 39218499). (3) Rev-erbα: human hCG93862 (34926094 to 34912094), mouse mCG15360 (105438925 to 105424925), rat rCG33292 (82492796 to 82478796). (4) Dbp: human NT_{—}011109.15 (c21417778 to 21402778), mouse NT_{—}078442.1 (59711 to 74711), rat NW_{—}047558.1 (5120734 to 5135734). (5) Per3: human NT_{—}021937.16 (1962822 to 1977822), mouse NT_{—}039268.2 (c4331528 to 4316528), rat NW_{—}047727.1 (c801656 to 8001956). (6)Per1: human NT_{—}010718.14 (c6905708 to 6890708), mouse NT_{—}039515.2 (65661216 to 65676216), rat rCG34390 (52960430 to 52974430). (7) Per2: human NT_{—}0051120.14 (c5136562 to 5121562), mouse NT_{—}039173.2 (c5833757 to 5818757), rat NW_{—}047817.1 (c6827703 to 6812703).

The base sequences of RORE, DBP binding site and E BOX, which are transcription control regions of the circadian rhythm control genes, are shown in a sequence table (sequence number 1 to sequence number 3, wherein “w” is a or t, “n” is any nucleotide, “d” is a or g, “r” is g or a, “k” is g or t, and “y” is t or c).

It will be noted that the expression peaks 6 the circadian rhythm control gene clusters (1) to (7) under normal conditions are, respectively, at 20 o'clock to 24 o'clock in terms of circadian time for the genes (1) or/and (2), at 4 o'clock to 8 o'clock as circadian time for the gene (3), at 6 o'clock to 10 o'clock as circadian time for the gene (4), at 8 o'clock to 12 o'clock as circadian time for the gene (5), at 10 o'clock to 14 o'clock as circadian time for the gene (6), and at 12 o'clock to 16 o'clock as circadian time for the gene (7).

Next, according to the invention, there is provided a DNA chip having at least the circadian rhythm control gene clusters sequenced therein. The DNA chip can be used, for example, for (1) a method for predicting a modulation of expression timings of the circadian rhythm gene clusters, (B) a method for detecting a modulation of circadian rhythm, (C) a method for selecting a circadian rhythm regulator; (D) screening of a circadian rhythm regulator, and (E) screening such as of medicines involving a modulation of a circadian rhythm as an ill effect.

(A) Method for Predicting a Modulation in Expression Timing of Circadian Rhythm Control Gene Clusters:

As stated hereinbefore, we have clarified the interrelation between the expression timing of circadian rhythm control gene cluster and the binding sites (RORE, DBP binding site, E-BOX) of transcription factor. Using the interrelation, it can be predicted how the expression timings of the circadian rhythm control gene clusters modulate. More particularly, when the modulation of an expression timing in a circadian rhythm control gene having a specific type of transcription factor binding site in a code region or an upstream sequence thereof is detected, the modulation of the expression timing in the circadian rhythm control gene having the transcription factor binding site can be detected.

For instance, if a modulation in expression timing of a gene (e.g. Bmal1) having RORE in a code region or an upstream sequence thereof is detected, a modulation in expression timing of other genes (Npas2, Rev-erbα, Dbp) having ROR E in a code region or an upstream sequence thereof can be predicted. Likewise, when a modulation in expression timing of a gene (e.g. Per3) having a DBP binding site in a code region or an upstream sequence thereof is detected, other genes (e.g. Per1, Rev-erbα and the like) having a DBP binding site in a code region or an upstream sequence thereof can be predicted.

Additionally, when a modulation in expression timing of a gene having E-BOX in a code region or an upstream sequence thereof is detected, other genes (e.g. Per1, Rev-erbα, Dbp) having E-BOX in a code region or an upstream site thereof can be predicted.

(B) Method of Detecting a Modulation of Circadian Rhythm:

The circadian rhythm is modulated in case where expression timings (i.e. a time of expression peak) of the circadian rhythm control gene clusters are shifted, for example, in a sampled cell, where no expression of a specified circadian rhythm control gene is observed or where an expression sequence of the circadian rhythm control gene clusters differs from a normal one. Thus, when DNA extracted from a cell sampled at every clock-time interval is, for example, interacted with DNA on a DNA chip (such as by hybridization), a modulation of circadian rhythm can be readily detected.

It will be noted that for diseases accompanying the modulation of circadian rhythm, mention is made, for example, of sleep disorder, awakening disorder, jet syndrome, insomnia, autonomic ataxia, depression, senile dementia, deterioration of physical balance through irregularities of life style involved by night work, shift work or the like, fatigues through irregularities of life style accompanied by autistic and the like. For diseases induced from the modulation of circadian rhythm, there is mentioned, for example, an increase in breast cancer incidence such as of nurses as would be caused by night work.

(C) Method for Selecting a Circadian Rhythm Regulator:

Where circadian rhythm is modulated, ready detection using a DNA chip of the invention is possible as to whether the modulation of the circadian rhythm is based on the expression abnormality of which gene is selected among circadian rhythm control gene clusters. Accordingly, where no expression of Per3 (having a DBP binding site in the sequence) is observed or where shift in expression timing of Per3 is detected, for example, selection of a circadian rhythm regulator acting on the Dbp binding site leads to an improvement in modulation of the circadian rhythm. Likewise, where no expression of Bmal1 (having RORE in a promoter site) is observed or where shift in expression timing of Bmal1 is detected, the modulation of the circadian rhythm can be improved by selection of a circadian rhythm regulator acting on RORE. This is true of other types of circadian rhythm control genes.

(D) Screening of Circadian Rhythm Regulators:

Using the DNA chip of the invention, substances capable of shifting expression timings of circadian rhythm control gene clusters, i.e. candidates for circadian rhythm regulator, can be comprehensively searched. For example, where the expression timing of Per3 gene is shifted forwardly (i.e where the expression timing is advanced), such a substance may become a potential candidate for circadian rhythm regulator in terms of a factor promoting the expression of Per3 gene or a substance acting directly or indirectly on DBP binding site. This is true of other types of circadian rhythm control genes.

(E) Screening of Substances Involving the Modulation of Circadian Rhythm as a Side Effect:

Comprehensive searches for all substances utilized as a medicine are performed using the DNA chip related to the invention, by which any substances accompanying the modulation of circadian rhythm as a side effect can be detected, for example.

Where a substance involving the modulation of circadian rhythm is detected, an administration timing of the substance can be determined. For instance, where such a substance is found, as a result of the screening, to be one which is able to shift the expression timing of Per3 gene, such a timing as not to shift the expression timing of the Per3 gene enables the side effect of the substance to be prevented. This is applicable to the cases of other types of circadian rhythm control genes.

The technical terms used herein are defined below.

The term “circadian rhythm control gene cluster” generically means genes related to a control mechanism of circadian rhythm in vivo. The term “circadian rhythm control gene” means genes related to a control mechanism of circadian rhythm.

The term “circadian time (CT)” means a time at which a light conditions initiating time is taken as 0 o'clock (CT 0). In contrast, the term “ordinary time” is one wherein one day starts from 0 a.m. and ends with 12 p.m.

The term “code region” means a region where genes on a base sequence are coded and a sequence covering from a transcription initiating point to a transcription completing point. The term “upstream sequence” means a sequence upstream of the transcription initiating point of the code region.

The term “circadian rhythm peripheral tissues” means tissues other than a circadian rhythms center (nucleus suprachiasmatica (SCN) of the brain's hypothalamus region, and includes, for example, heart, lung, liver, stomach, spleen and kidney.

The term “circadian rhythm regulator” means those regulators covering medicines, other substances and compositions having the action of improving the modulation of circadian rhythm.

As will be apparent from the foregoing, according to the invention, the expression timings and sequences of circadian rhythm control gene clusters and abnormalities thereof can be detected.

The invention is more particularly decribed by way of examples.

### BRIEF DESCRIPTION OF THE DRAWINGS

FIG. 1 is a view showing the results of real time RT-PCR;

FIG. 2 is a schematic view showing expression timings of circadian rhythm control gene clusters;

FIG. 3 is a vow showing a position or location of a transcription control site in individual circadian rhythm control genes; and

FIG. 4 is a schematic view showing an expression control mechanism of circadian rhythm control gene clusters.

Sequence Table: 200407061434313900_A163 0490491101 12004199122 AAA0.app

### DETAILED DESCRIPTION OF THE INVENTION

### EXAMPLE 1

In Example 1, expression peaks at every clocktime interval of circadian rhythm control genes in a circadian rhythm peripheral tissues were checked. The procedure was as follows.

Initially, a circadian rhythm of a mouse used for an experiment was synchronized. More particularly, a mouse was bred over two weeks in a room which was kept under light conditions from 8 to 20 o'clock and under dark conditions from 20 to 8 o'clock of the next day so that the circadian rhythm of the mouse was synchronized. It will be noted that for the experiment, ICR mouse (male, five-week old) purchased from Nihon SLC kabushikikaisha was used.

Net, the heart, lung, liver, stomach, spleen and testis were, respectively, sampled from the mouse at given time intervals as a circadian rhythm peripheral tissue and immediately frozen with liquid nitrogen. It will be noted that the sampling time for the respective internal organs was so set as to be every four hours from 8 o'clock determined immediately after the synchronization of the circadian rhythm (8 o'clock, 12 o'clock, 16 o'clock, 20 o'clock, 24 o'clock and 4 o'clock of the next day).

Thereafter, the sampled organs were, respectively, homogenized, followed by extraction of total RNA from individual organs by use of Promega Total SV RNA Isolation Kit (made by Promega Corporation). Next, quantitative real time RT-PCR (quantitative real time reverse transcription polymerase chain reaction) was carried out to measure expressed amounts of circadian rhythm control genes.

The thus measured circadian rhythm control genes were 14 in number as indicated below. Bal1, Npas2, Rev-erbα, Dbp, Rev-erbβ, Per3, Per1, Per2, Cry1, Cry2, Clock, Cklδ, Ckl ε and Tim.

It will be noted that quantitative real time RTPCR used was ABI PRISSM 7000 (made by Applied Biosystems, Ltd.) For PCR, 40 cycles of 50° C., 2 minutes/95° C., 10 minutes/(95° C., 15 seconds/60° C., 1 minute) were carried out using SYBR Green PCR Master Mix (ABI Inc.). The primers used are indicated below.

Bal1 (sense primer: sequence No. 4, antisense primer: sequence No. 5), Npas2 (sense primer: sequence No. 6, antisense primer: sequence No. 7), Rev-erbα (sense primer: sequence No. 8, antisense primer: sequence No. 9), Dbp (sense primer: sequence No. 10, antisense primer: sequence No. 11), Rev-erbβ (sense primer: sequence No. 12, antisense primer: sequence No. 13), Per3 (sense primer: sequence No. 14, antisense primer: sequence No. 15), Per1 (sense primer: sequence No. 16, antisense primer: sequence No. 17), Per2 (sense primer: sequence No. 18, antisense primer: sequence No. 19), Cry1 (sense primer: sequence No. 20, antisense primer: sequence No. 21), Cry2 (sense primer: sequence No. 22, antisense primer: sequence No. 23), Clock (sense primer: sequence No. 24, antisense primer: sequence No. 25), Ckl δ (sense primer: sequence No. 26, antisense primer: sequence No. 27), Ckl ε (sense primer: sequence No. 28, antisense primer: sequence No. 29), and Tim (sense primer: sequence No. 30, antisense primer: sequence No. 31).

In the above experiment, the expression level of the respective genes is a relative value calculated based on the expressed amount of a housekeeping gene. First, transcription products of β-actin and G3PDH were simultaneously amplified as selected from the total RNA extracted at the time of reverse transcription PCR in the course of the experimental procedure, followed by synthesis of cDNA of β-actin and G3PDH. The β-actin and G3PDH were both a housekeeping gene, with expression levels being substantially constant. The expression levels of the respective genes were compared with the expression level of β-actin or G3PDH, from which a relative expression level was calculated. It is to be noted that for the calculation of expression level, two types of housekeeping genes (β-actin and G3PDH) were used to obtain expression levels of individual genes, and the accuracy of the expression level of individual genes was increased by correcting and adjusting errors involved in the case using β-actin and also in the case using G3PDH.

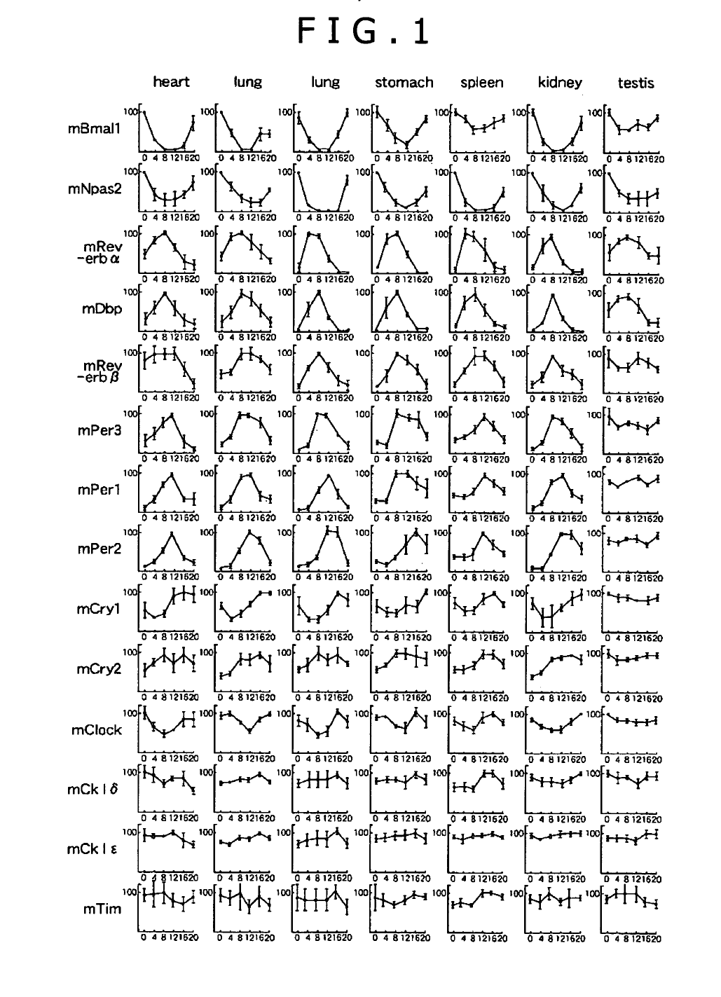

The results are shown in FIG. 1 in which the expression levels of the respective genes are shown for heart, lung, liver, stomach, spleen, kidney and testis. The symbol “m” in the genes (e.g. initially occurring “m” in “mBal1”) means mammalian. In each graph, the abscissa indicates a circadian time (CT), and the ordinate indicates a relative expression level (%). As to the circadian time (CT), CT0 to CT12 are under light conditions (subjective daytime) and CT12 to CT24 are under dark conditions (subjective night).

As shown in FIG. 1, the circadian change of the circadian rhythm control gene expression was observed for eight genes among 14 genes in total (Bmal1, Npas2, Rev-erbα, Rev-erbβ, Dbp, Per3, Per1 and Per2) in all the organs except for testis.

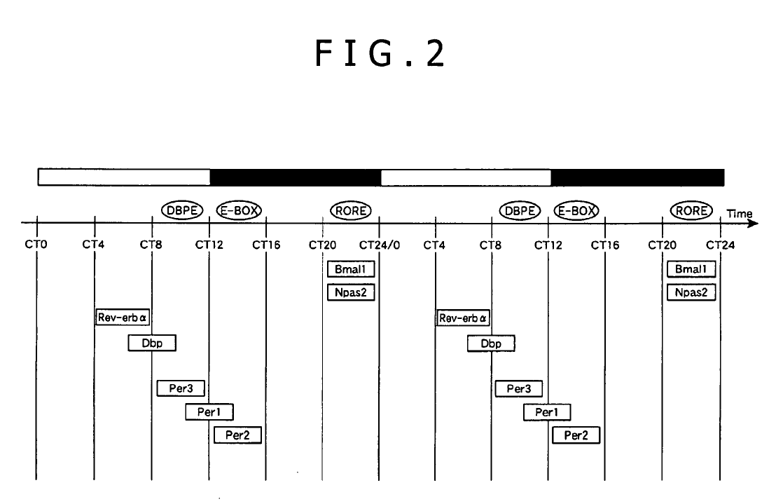

In FIG. 2, expression peaks of the respective genes are schematically shown. Outline portions at the uppermost portion of FIG. 2 indicate light conditions (CT0 to CT12), and solid portions indicate dark conditions (CT12 to CT24). The abscissa indicates circadian time (CT). A bind timing predicting time of a transcription factor relative to “DBPE”, “E-BOX” and “RORE” acting as a transcription factor binding region is indicated on the abscissa.

As shown in FIG. 2, the expression peaks of individual genes were at CT20 to CT0 for Bmal1 and Npas2, CT4 to CT8 for Rev-erbα, CT6 to CT10 for Dbp, CT8 to CT12 for Per3, CT10 to CT14 for Per1, and CT12 to CT16 for Per2. The expression peaks of the respective circadian rhythm control genes in individual organs were found to be substantially at the same clock time.

The above results suggested that genes controlling the circadian rhythm (molecular biological clock) of circadian rhythm peripheral tissues were mainly eight (Bmal1, Npas2, Rev-erbα, Rev-erbβ, Dbp, Per3, Per1, Per2) in number among the circadian rhythm control genes.

Further, the above results indicate that the sequence of expression of the circadian rhythm control genes in the respective circadian rhythm peripheral tissues has a certain pattern. More particularly, it is shown that in the course of one day of CT0 to CT24 at normal time, the circadian rhythm control gene cluster is expressed in the order of Rev-erbα, Dbp, Per3, Per1 and Per2 in time series, and then Bmal1 and Npas2 express at the same hour.

In addition, the results suggest the possibility that the circadian rhythm peripheral tissues as a whole reset circadian rhythms according to a similar mechanism. That is, the fact that the expression peaks of the circadian rhythm control genes appear substantially at the same clock time for whole circadian rhythm peripheral tissue suggests the possibility that the respective circadian rhythm peripheral tissues reset circadian rhythms based on the circadian rhythm of circadian rhythm center (SCN) according to similar mechanisms, respectively.

Besides, the results on testis in FIG. 1 suggest the possibility of Per1 that plays a role other than the formation of circadian rhythm. In this experiment, it was found that with testis, weak circadian changes were observed for four genes (Bmal1, Npas2, Rev-erbα and Dbp) as shown in FIG. 1. On the other hand, with Per1 gene, although no circadian change of expression of Per1 gene was observed, the expressed amount or level was higher than those of other circadian rhythm control genes. Thus, the results of this experiment suggest that in testis, Per1 plays a role other than the formation of circadian rhythm, e.g. the control of occurrence upon spermatogenesis.

### EXAMPLE 2

In Example 2, using NCBI database and Celera database system, transcription control regions (E-BOX, DBP binding region and RORE) of circadian clock control genes existing in eight circadian clock control genes (Bmal1, Npas2, Rev-erbα, Rev-erbβ, Dbp, Per3, Per1 and Per2) were searched.

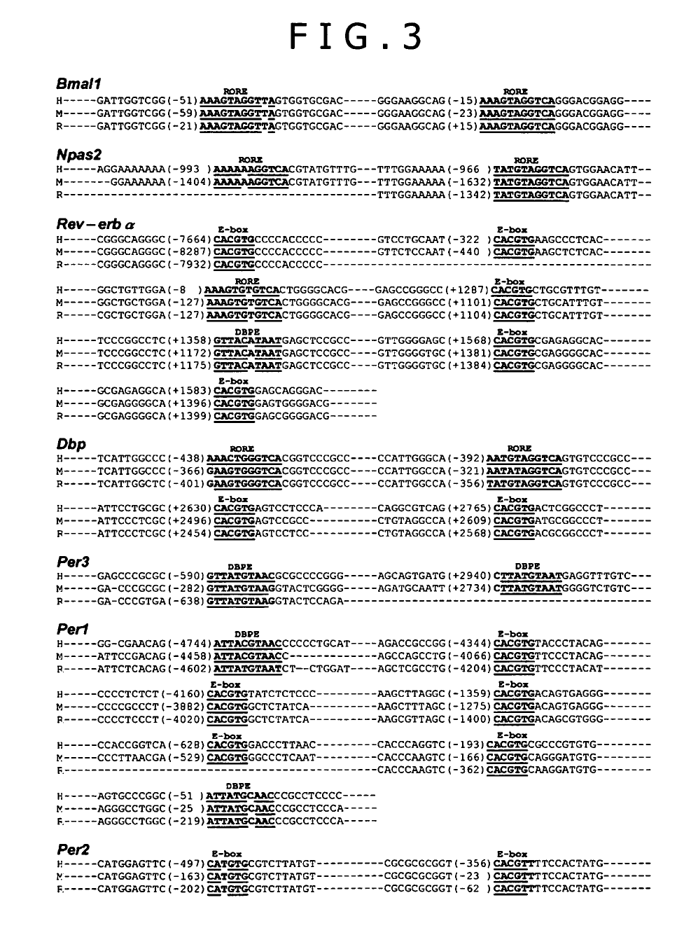

The results of the search are as follows (see FIG. 3).

(1)RORE existed in the promoter region of Bmal1 gene.

(2)RORE existed in the promoter region of Npas2 gene.

(3) One RORE, one DBP binding site and five E-BOXs existed in the Rev-erbα gene.

(4)Two ROREs and two E-BOXs existed in the Dbp gene.

(5)The DBP binding site existed in the promoter region of the Per3 gene within the gene thereof.

(6)Five E-BOXs and DBP binding region existed in the promoter region of the Per1 gene.

(7)E-BOX existed in the promoter region of the Per2 gene. The E-BOX (CACGTT) in the Per2 gene differs in sequence from typical E-BOX (CACGTG).

These results suggest the existence of the following circadian rhythm control mechanisms.

(1) The expression of Bmal1 gene and (2) the expression of Npas2 gene are controlled through transcription factors (Rev-erb and the like) bound to RORE. On the other hand, Bmal1 gene expresses BMAL1 (protein). The thus expressed BMAL1 (protein) binds with CLK (protein) to form a CLK-BMAL1 complex. The CLK-BMAL1 complex promotes the expression of E-BOX-bearing genes (Rev-erbα gene, Dbp gene, Per1 gene and Per2 gene) in a promoter region. It will be noted that Npas2 gene expresses NPAS2 (protein).

(3) It is assumed that the expression of Reverbα gene undergoes control of the transcription factors (Rev-erb and the like) bound to RORE, the transcription factors (DBP and the like) bound to the DBP binding site and the transcription factor (CLK-BMAL1 complex) bound to E-BOX. More particularly, it is assumed that the expression of Rev-erbα gene undergoes control in expression level by a feedback system. On the other hand, the Rev-erbα gene expresses Rev-erbα (protein). The REV-ERBα (protein) serves as a transcription factor and controls the expression of RORE-bearing genes (Bmal1 gene and Npas2 gene).

(4) The expression of Dbp gene is assumed to undergo control of the transcription factors (Rev-erb and the like) bound to RORE and the transcription factor (CLK-BMAL1 complex) bound to E-BOX. On the other hand, the Dbp gene expresses DBP (protein). DBP serves as a transcription factor and binds to a DBP binding site to promote the expression of DBP binding site-bearing genes (Rev-erbα gene, Per3 gene and Per1 gene).

(5) The expression of Per3 gene is promoted by means of the transcription factor bound to the DBP binding site such as DBP.

(6) The expression of Pet gene undergoes control of the transcription factor bound to the DBP binding site such as DBP and the transcription factor (CLK-BMAL1 complex) bound to E-BOX.

(7) The expression of Per2 gene undergoes control of the transcription factor (CLK-BMAL1 complex) bound to E-BOX.

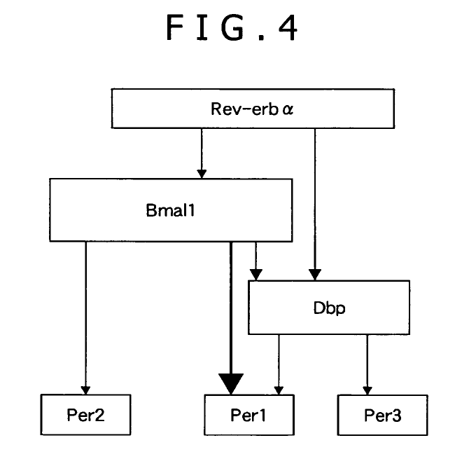

In summary, as scheatically shown in FIG. 4, the circadian clock control gene suggests the possibility of permitting Rev-erbα gene, Bmal1 gene, Dbp gene, and Per gene to be expressed in this order. More particularly, there is suggested such possibility that the proteins expressed from the respective genes control downstream gene expression. The order of the expression is substantially coincident with the circadian expression timing indicated in Example 1 (see FIG. 2).

It will be noted that in FIG. 4, the arrow indicating the action, on the Rev-erbα, of the proteins expressed from the circadian clock control gene is omitted. It is assumed that in Example 1 (FIG. 2), the reason why the expression timing of Rev-erbα exists between Bmal1/Npas2 and Dbp is that the Rev-erbα gene has a plurality of transcription factors (one RORE, one DBP binding sites and five E-BOXs), or the expression of Rev-erbα gene is controlled due to a complicated feedback system. In FIG. 2, the expression timings of the Dbp gene expressing at CT6 to CT10 and also of the Bmal1 gene at next CT20 to CT24 are controlled by the transcription product (REV-ERBα) of the Rev-erbα gene expressing at CT4 to CT8.

Besides, it is assumed that the variations in expression timing of Per3 gene, Per1 gene, and Per2 gene result from differences in type, number and sequence of transcription factor binding sites or the existence of a complicated control system.

The circadian rhythm control genes according to the invention may be applicable not only to DNA chips, but also, for example, to cultured cells having, at least, the circadian rhythm control gene clusters. The cultured cells have the possibility of application to a screening method for substances involving the modulation, as an ill effect, of circadian rhythm. From the foregoing, the circadian rhythm control gene clusters according to the invention are industrially useful.

While the preferred embodiments of the present invention have been described using the specific terms, such description is for illustrative purposes only, and it is to be understood that changes and variations may be made without departing from the spirit or scope of the following claims.

What is claimed is:

1. A circadian rhythm control gene cluster in which after expression of the following genes (1) or/and (2), the following genes (3) to (7) start to express sequentially and which contains all the genes (1) to (7) or a plurality of genes selected from the genes (1) to (7):
(1)Bmal1 gene having RORE in a code region or an upstream sequence thereof;
(2) Npas2 gene having RORE in an upstream sequence of a code region;
3( ) Rev-erbα gene having RORE, DBP binding site and E-BOS in a code region and an upstream sequence thereof;
4( ) Dbp gene having RORE and E-BOX in a code region or an upstream sequence thereof;
5( ) Per3 gene having a DBP binding site in a code region or an upstream sequence thereof;
6( ) Per1 gene having a DBP binding site in an upstream sequence of a code region; and
7( ) Per2 gene having E-BOX in an upstream sequence of a code region.

2. The circadian rhythm control gene cluster according to claim 1, wherein when standardized on 16 o'clock in terms of circadian time, the circadian rhythm control gene cluster expresses in the following sequence of:
(a) a gene having RORE in a code region or an upstream sequence thereof;
b( ) a gene having a DBP binding site in a code region or an upstream sequence thereof; and
c( ) a gene having E-BOX in a code region or an upstream sequence thereof.

3. The circadian rhythm control gene cluster according to claim 1, wherein the expression appears at circadian rhythm peripheral tissues.

4. The circadian rhythm control gene cluster according to claim 1, wherein expression peaks of the circadian rhythm control gene cluster (1) to (7) under normal conditions are, respectively,
at 20 o'clock to 24 o'clock in terms of circadian time for the genes (1) or/and (2),
at 4 o'clock to 8 o'clock as circadian time for the gene (3),
at 6 o'clock to 10 o'clock as circadian time for the gene (4),
at 8 o'clock to 12 o'clock as circadian time for the gene (5),
at 10 o'clock to 14 o'clock as circadian time for the gene (6), and
at 12 o'clock to 16 o'clock as circadian time for the gene (7).

5. A DNA chip comprising, at least, the circadian rhythm control gene cluster defined in claim 1 being sequentially arranged.

6. A method for predicting modulation in expression timing of a circadian rhythm control gene cluster, by using the DNA chip defined in claim 5.

7. The method according to claim 6, wherein modulation in expression timing of a gene having RORE in a code region or an upstream sequence thereof is detected to predict modulation in expression timing of other gene having RORE in the code region or the upstream sequence thereof.

8. The method according to claim 6, wherein modulation in expression timing of a gene having a DBP binding site in a code region or an upstream sequence thereof is detected to predict modulation in expression timing of other gene having the DBP binding site in the code region or the upstream sequence thereof.

9. The method according to claim 6, wherein modulation in expression timing of a gene having E-BOX in a code region or an upstream sequence thereof is detected to predict modulation in expression timing of other gene having E-BOX in the code region or the upstream sequence thereof.

10. A method for detecting modulation of a circadian rhythm using the DNA chip defined in claim 5.

11. The method according to claim 10, wherein sleep disorder is detected.

12. The method according to claim 10, wherein insomnia is detected.

13. The method according to claim 10, wherein jet syndrome is detected.

14. A method for selecting a circadian rhythm regulator using the DNA chip defined in claim 5.

15. A method for screening a substance acting on any or a plurality of RORE, a DBP binding site and E-BOX using the DNA chip defined in claim 5.

16. The method according to claim 15, wherein said substance consists of a circadian rhythm regulator.

17. The method according to claim 15, wherein said substance regulates modulation of a circadian rhythm.


SEQUENCE LISTING (52 sequences)

SEQ ID NO: 1 - DNA, 11 bp, Artificial Sequence
  Description of Artificial Sequence Synthetic RORE sequence
wawntdggtca

SEQ ID NO: 2 - DNA, 10 bp, Artificial Sequence
  Description of Artificial Sequence Synthetic DBPE sequence
rtkaygtaay

SEQ ID NO: 3 - DNA, 6 bp, Artificial Sequence
  Description of Artificial Sequence Synthetic E-BOX sequence
cacgtg

SEQ ID NO: 4 - DNA, 22 bp, Artificial Sequence
  Description of Artificial Sequence Synthetic sense primer of Bal1
gcagtgccactgactaccaaga

SEQ ID NO: 5 - DNA, 20 bp, Artificial Sequence
  Description of Artificial Sequence Synthetic antisense primer of Bal1
tcctggacattgcattgcat

SEQ ID NO: 6 - DNA, 24 bp, Artificial Sequence
  Description of Artificial Sequence Synthetic sense primer of Npas2
gtatgcacagagccaagtgatgtt

SEQ ID NO: 7 - DNA, 22 bp, Artificial Sequence
  Description of Artificial Sequence Synthetic antisense primer of Npas2
tgctcactgtgcagagatgttg

SEQ ID NO: 8 - DNA, 20 bp, Artificial Sequence
  Description of Artificial Sequence Synthetic sense primer of Rev-erb alpha
cgttcgcatcaatcgcaacc

SEQ ID NO: 9 - DNA, 18 bp, Artificial Sequence
  Description of Artificial Sequence Synthetic antisense primer of Rev-erb alpha
gatgtggagtaggtgagg

SEQ ID NO: 10 - DNA, 25 bp, Artificial Sequence
  Description of Artificial Sequence Synthetic sense primer of Dbp
aatgacctttgaacctgatcccgct

SEQ ID NO: 11 - DNA, 25 bp, Artificial Sequence
  Description of Artificial Sequence Synthetic antisense primer of Dbp
gctccagtacttctcatccttctgt

SEQ ID NO: 12 - DNA, 20 bp, Artificial Sequence
  Description of Artificial Sequence Synthetic sense primer of Rev-erb beta
acggattcccaggaacatgg

SEQ ID NO: 13 - DNA, 20 bp, Artificial Sequence
  Description of Artificial Sequence Synthetic antisense primer of Rev-erb beta
cctccagtgttgcacaggta

SEQ ID NO: 14 - DNA, 22 bp, Artificial Sequence
  Description of Artificial Sequence Synthetic sense primer of Per3
ctgctccaactcagcttccttt

SEQ ID NO: 15 - DNA, 23 bp, Artificial Sequence
  Description of Artificial Sequence Synthetic antisense primer of Per3
ttagacagcaaggctctggttct

SEQ ID NO: 16 - DNA, 25 bp, Artificial Sequence
  Description of Artificial Sequence Synthetic sense primer of Per1
caggctaaccaggaatattaccagc

SEQ ID NO: 17 - DNA, 25 bp, Artificial Sequence
  Description of Artificial Sequence Synthetic antisense primer of Per1
cacagccacagagaaggtgtcctgg

SEQ ID NO: 18 - DNA, 20 bp, Artificial Sequence
  Description of Artificial Sequence Synthetic sense primer of Per2
ggcttcaccatgcctgttgt

SEQ ID NO: 19 - DNA, 23 bp, Artificial Sequence
  Description of Artificial Sequence Synthetic antisense primer of Per2
ggagttatttcggaggcaagtgt

SEQ ID NO: 20 - DNA, 25 bp, Artificial Sequence
  Description of Artificial Sequence Synthetic sense primer of Cry1
cccaggcttttcaaggaatggaaca

SEQ ID NO: 21 - DNA, 25 bp, Artificial Sequence
  Description of Artificial Sequence Synthetic antisense primer of Cry1
tctcatcatggtcatcagacagagg

SEQ ID NO: 22 - DNA, 23 bp, Artificial Sequence
  Description of Artificial Sequence Synthetic sense primer of Cry2
gggactctgtctattggcatctg

SEQ ID NO: 23 - DNA, 20 bp, Artificial Sequence
  Description of Artificial Sequence Synthetic antisense primer of Cry2
gtcactctagcccgcttggt

SEQ ID NO: 24 - DNA, 22 bp, Artificial Sequence
  Description of Artificial Sequence Synthetic sense primer of Clock
cctatcctaccttggccacaca

SEQ ID NO: 25 - DNA, 20 bp, Artificial Sequence
  Description of Artificial Sequence Synthetic antisense primer of Clock
tcccgtggagcaacctagat

SEQ ID NO: 26 - DNA, 20 bp, Artificial Sequence
  Description of Artificial Sequence Synthetic sense primer of Ck1 delta
ccagcctggaagacctgttc

SEQ ID NO: 27 - DNA, 18 bp, Artificial Sequence
  Description of Artificial Sequence Synthetic antisense primer of Ck1 delta
tggccagcccaaagtcaa

SEQ ID NO: 28 - DNA, 21 bp, Artificial Sequence
  Description of Artificial Sequence Synthetic sense primer of Ck1 epsilon
ggatgtgaagcccgacaactt

SEQ ID NO: 29 - DNA, 20 bp, Artificial Sequence
  Description of Artificial Sequence Synthetic antisense primer of Ck1 epsilon
tctcgacggctttgctcaat

SEQ ID NO: 30 - DNA, 18 bp, Artificial Sequence
  Description of Artificial Sequence Synthetic sense primer of Tim
acatgtgggcaatggctt

SEQ ID NO: 31 - DNA, 21 bp, Artificial Sequence
  Description of Artificial Sequence Synthetic antisense primer of Tim
ctgctccacaaagtgaaaggt

SEQ ID NO: 32 - DNA, 62 bp, Homo sapiens
gattggtcggaaagtaggttagtggtgcgacgggaaggcagaaagtaggtcagggacggagg

SEQ ID NO: 33 - DNA, 62 bp, Mus sp.; identical to SEQ ID NO: 32

SEQ ID NO: 34 - DNA, 62 bp, Rattus sp.; identical to SEQ ID NO: 32

SEQ ID NO: 35 - DNA, 62 bp, Homo sapiens
aggaaaaaaaaaaaaaggtcacgtatgtttgtttggaaaaatatgtaggtcagtggaacatt

SEQ ID NO: 36 - DNA, 60 bp, Mus sp.
ggaaaaaaaaaaaaggtcacgtatgtttgtttggaaaaatatgtaggtcagtggaacatt

SEQ ID NO: 37 - DNA, 31 bp, Rattus sp.
tttggaaaaatatgtaggtcagtggaacatt

SEQ ID NO: 38 - DNA, 191 bp, Homo sapiens
cgggcagggccacgtgccccacccccgtcctgcaatcacgtgaagccctcacggctgttggaaaagtgtgtcactggggcacggagccgggcccacgtgctgcgtttgttcccggcctcgttacataatgagctccgccgttggggagccacgtgcgagaggcacgcgagaggcacacgtggagcagggac

SEQ ID NO: 39 - DNA, 192 bp, Mus sp.
cgggcagggccacgtgccccacccccgttctccaatcacgtgaagctctcacggctgctggaaaagtgtgtcactggggcacggagccgggcccacgtgctgcatttgttcccggcctcgttacataatgagctccgccgttggggtgccacgtgcgaggggcacgcgaggggcacacgtggagtggggacg

SEQ ID NO: 40 - DNA, 166 bp, Rattus sp.
cgggcagggccacgtgccccaccccccgctgctggaaaagtgtgtcactggggcacggagccgggcccacgtgctgcatttgttcccggcctcgttacataatgagctccgccgttggggtgccacgtgcgaggggcacgcgaggggcacacgtggagcggggacg

SEQ ID NO: 41 - DNA, 114 bp, Homo sapiens
tcattggcccaaactgggtcacggtcccgccccattgggcaaatgtaggtcagtgtcccgccattcctgcgccacgtgagtcctcccacaggcgtcagcacgtgactcggccct

SEQ ID NO: 42 - DNA, 112 bp, Mus sp.
tcattggcccgaagtgggtcacggtcccgccccattggccaaatataggtcagtgtcccgccattccctcgccacgtgagtccgccctgtaggccacacgtgatgcggccct

SEQ ID NO: 43 - DNA, 112 bp, Rattus sp.
tcattggctcgaagtgggtcacggtcccgccccattggccatatgtaggtcagtgtcccgccattccctcgccacgtgagtcctccctgtaggccacacgtgacgcggccct

SEQ ID NO: 44 - DNA, 60 bp, Homo sapiens
gagcccgcgcgttatgtaacgcgccccgggagcagtgatgcttatgtaatgaggtttgtc

SEQ ID NO: 45 - DNA, 59 bp, Mus sp.
gacccgcgcgttatgtaaggtactcggggagatgcaattcttatgtaatggggtctgtc

SEQ ID NO: 46 - DNA, 29 bp, Rattus sp.
gacccgtgagttatgtaaggtactccaga

SEQ ID NO: 47 - DNA, 188 bp, Homo sapiens
ggcgaacagattacgtaacccccctgcatagaccgccggcacgtgtaccctacagcccctctctcacgtgtatctctcccaagcttaggccacgtgacagtgagggccaccggtcacacgtggacccttaaccacccaggtccacgtgcgcccgtgtgagtgcccggcattatgcaacccgcctcccc

SEQ ID NO: 48 - DNA, 178 bp, Mus sp.
attccgacagattacgtaaccagccagcctgcacgtgttccctacagccccgccctcacgtggctctatcaaagctttagccacgtgacagtgagggcccttaacgacacgtgggccctcaatcacccaagtccacgtgcagggatgtgagggcctggcattatgcaaccgcctccca

SEQ ID NO: 49 - DNA, 160 bp, Rattus sp.
attctcacagattatgtaatctctggatagctcgcctgcacgtgttccctacatcccctccctcacgtggctctatcaaagcgttagccacgtgacagcgtgggcacccaagtccacgtgcaaggatgtgagggcctggcattatgcaacccgcctccca

SEQ ID NO: 50 - DNA, 52 bp, Homo sapiens
catggagttccatgtgcgtcttatgtcgcgcgcggtcacgttttccactatg

SEQ ID NO: 51 - DNA, 52 bp, Mus sp.; identical to SEQ ID NO: 50

SEQ ID NO: 52 - DNA, 52 bp, Rattus sp.; identical to SEQ ID NO: 50